(19) United States
(12) Patent Application Publication
(10) Pub. No.: US 2025/0179058 A1
(43) Pub. Date: Jun. 5, 2025

(54) THIOPHENE CONFIGURABLE AMORPHOUS SMALL MOLECULAR HOLE TRANSPORTING MATERIAL FOR EFFICIENT AND STABLE PEROVSKITE SOLAR CELL

(71) Applicant: The Regents of the University of California, Oakland, CA (US)

(72) Inventors: Yang YANG, Oakland, CA (US); Dong MENG, Oakland, CA (US)

(73) Assignee: The Regents of the University of California, Oakland, CA (US)

(21) Appl. No.: 18/540,357

(22) Filed: Dec. 14, 2023

Related U.S. Application Data
(63) Continuation of application No. PCT/US2023/082166, filed on Dec. 1, 2023.
(60) Provisional application No. 63/429,780, filed on Dec. 2, 2022.

(51) Int. Cl. C07D 409/14; H10K 50/15; H10K 85/60

(52) U.S. Cl. CPC C07D 409/14; H10K 50/15; H10K 85/633; H10K 85/655

(57) ABSTRACT

A spiro-thiophene compound includes a centroid core having a plurality of benzene rings, and a plurality of thiophene molecules covalently bonded to a carbon atom of the plurality of benzene rings of the centroid core to form a centroid-thiophene molecule.

### CROSS REFERENCE TO RELATED APPLICATION

The present patent application is a continuation of International Application No. PCT/US2023/082166, filed on Dec. 1, 2023, which claims priority benefit from U.S. Provisional Patent Application No. 63/429,780 filed on Dec. 2, 2022, the entire content of which is incorporated herein by reference.

### FIELD OF THE INVENTION

The field of the currently claimed embodiments of this invention relates generally to charge transporting materials and, more specifically to a thiophene configurable charge transporting material.

### BACKGROUND

Hole transporting materials (HTMs) are used in many areas of electronics including energy harvesting solar cells and organic light emitting diodes. HTMs are used in perovskite solar cells both for the n-i-p and the p-i-n device structures. HTMs can also be used to replace the liquid electrolyte of conventional dye-sensitized solar cells to make solid-state dye-sensitized solar cell (ss-DSSC) devices.

### BRIEF DESCRIPTION OF THE DRAWINGS

The present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1A shows a chemical formula of an example Spiro-8Th used as a hole transport layer (HTL), according to an embodiment of the present invention;

FIG. 1B shows a chemical formula of an example Spiro-OMeTAD used as the hole transport layer (HTL), according to another embodiment of the present invention;

FIG. 2 is a plot of Normalized PCE (%) versus elapsed time (hour) showing the photo-stability (at about 90±10 mW cm⁻²) for both the Spiro-8Th and the Spiro-OMeTAD, according to an embodiment of the present invention;

FIG. 3 is a plot of Normalized PCE (%) versus elapsed time (hour) showing the thermal stability (at about 85° C.) for both the Spiro-8Th and the Spiro-OMeTAD, according to an embodiment of the present invention;

FIG. 4 is a plot of the current density (mA/cm2) versus the voltage (V) or (J-V) for both forward 11-1 Spiro-8Th F and reverse 11-1 Spiro-8Th R scan directions, according to an embodiment of the present invention;

FIG. 5 is a plot of current density J (in mA/cm2) versus voltage (V) of a perovskite solar cell using Cl-substituted Spiro-8Th as the HTL, according to an embodiment of the present invention;

FIG. 6A is a schematic diagram of a basic structure of a perovskite (PVSK) solar cell highlighting the layered components from a transparent substrate to an electrode, according to an embodiment of the present invention;

FIG. 6B is a schematic diagram of a normal structure of a perovskite (PVSK) solar cell with an additional passivation layer to enhance performance and stability, according to another embodiment of the present disclosure;

FIG. 6C is a schematic diagram of a normal structure of a perovskite (PVSK) solar cell featuring both electron blocking and passivation layers for improved efficiency and longevity, according to yet another embodiment of the present invention;

FIG. 7 is a schematic diagram of an inverted structure of a perovskite (PVSK) device with hole-blocking layer and passivation layer, according to an embodiment of the present invention;

FIG. 8A is a schematic diagram of a perovskite (PVSK) photonics device using a charge transport layer based on Spiro-8Th, according to an embodiment of the present invention;

FIG. 8B is a schematic diagram of perovskite (PVSK) light emitting device (LED) using a charge transport layer based on Spiro-8Th, according to an embodiment of the present invention;

FIG. 8C is a schematic diagram of perovskite (PVSK) X-ray detector using a charge transport layer based on Spiro-8Th, according to an embodiment of the present invention;

FIG. 9 is schematic diagram of a structure of a tandem device 900 using Spiro-8Th in the hole transport layer, according to an embodiment of the present invention;

FIG. 10 is a plot of current density J (in mA/cm²) versus voltage (V) of a perovskite (PVSK) solar cell using Cl-substituted Spiro-8Th as the HTL, according to an embodiment of the present invention; and

FIG. 11 shows an example of a small molecule spiro-8Th and polymer molecule Spiro-8Th in HTL which substituents can be tailored to meet desired applications, according to embodiments of the present invention.

### DETAILED DESCRIPTION

Various embodiments of the present invention describe a series of relatively inexpensive and stable small molecules, Spiro-8Th, by altering the structure of spiro substitution on thiophene. This technique achieves the highest recorded power conversion efficiency (PCE) of about 23.77% in a perovskite solar cell for small molecular charge-transfer materials while maintaining high thermal and photochemical stability.

For single-junction conventional electron transport layer (ETL), intrinsic absorber layer or hole-transport layer (HTL) (n-i-p) perovskite solar cells (PSCs), power conversion efficiencies (PCEs) as high as 25.7% can be achieved. These PCEs approach the PCEs of cutting-edge crystalline-silicon solar cells. Due to the undoped HTL and the development of highly crystalline perovskite films, inverted (p-i-n structure) devices including a deposition sequence of hole-transport layer (p), intrinsic absorber layer (i) and electron-transport layer (n) have shown better stabilities and lifetimes.

Various embodiments of the current invention are discussed in detail below. In describing embodiments, specific terminology is employed for the sake of clarity. However, the invention is not intended to be limited to the specific terminology so selected. A person skilled in the relevant art will recognize that other equivalent components and other methods can be employed without departing from the broad concepts of the current invention. All references cited anywhere in this specification, including the Background and Detailed Description sections, are incorporated by reference as if each had been individually incorporated.

Interfacial layers, such as ETL and HTL, play a significant role in the efficiency and stability of PSCs. A good interfacial layer provides sufficient charge collection efficiency, form an ohmic contact with an electrode, and protect the unstable perovskite (PVSK) active layer. The ETL or HTL can have a suitable work function, high carrier mobility, and good stability. In addition, for the inverted structure, the HTL-interfacial layer has received additional investigation due to the increased infection on the perovskite topping layer growth.

The thickness of the HTL in p-i-n devices (below about 20 nm) is less than the thickness of HTL in n-i-p devices (over about 50 nm). This leads to reduced transporting length that allows the HTL with lower hole mobility to still work well in p-i-n devices. The inverted configuration, however, may tend to impose higher requirements for the solubility of the HTL as it may be easily damaged by the dimethylformamide (DMF) or dimethyl sulfoxide (DMSO) containing precursor solutions of perovskite. Another factor to consider is that incident light passes through the HTL first in p-i-n architectures, whereas the incident light first encounters the perovskite layer in n-i-p devices. This may pose further restrictions to the absorption range of HTL in p-i-n devices to ensure maximum light absorption of the perovskite layer. Lastly, the HTL should not be highly hydrophobic as this will introduce additional challenges during the coating process.

Compared with inorganic HTL, which is typically metal oxides, organic HTL features low-temperature solution processing capability, superior mechanical properties, molecular structural tunability, and hydrophobicity. Most of the metal oxides demand high-temperature annealing, thus limiting their application. In contrast, organic hole transporting materials, either small molecules or polymers, are soluble in organic solvents such as chloroform, chlorobenzene, and dichloromethane, and allow for low-temperature processing. Moreover, the secondary bonds constructing the molecular packing in thin films render superior plasticity, ductility, or softness. This allows for wide application of organic hole transporting materials in flexible devices where brittle and rigid inorganic hole transporting materials often fail to achieve unless nanostructures are involved.

The perovskite (PVSK) solar cell application of Spiro-8Th: Devices are fabricated incorporating Spiro-8Th or Spiro-OMeTAD for HTL with SnO₂/FAPbI₃/Spiro-8Th or Spiro-OMeTAD/Au architecture (n-i-p).

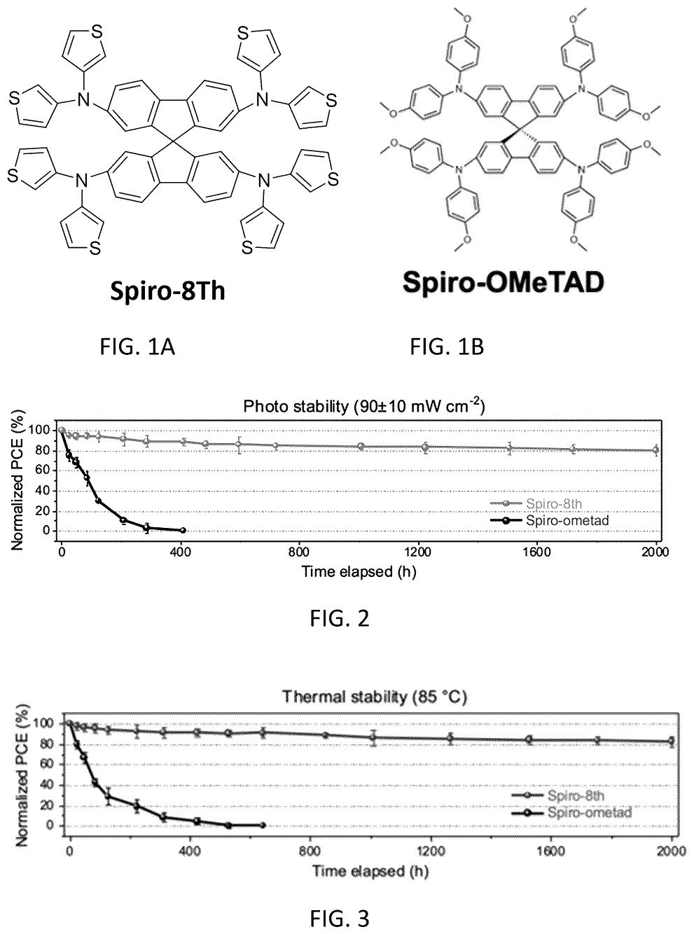

FIG. 1A shows a chemical formula of an example Spiro-8Th used as the hole transport layer (HTL), according to an embodiment of the present invention. FIG. 1B shows a chemical formula of an example Spiro-OMeTAD used as the hole transport layer (HTL), according to another embodiment of the present invention.

Due to higher hole mobility and superior charge extraction capability, distinct improvements of open-circuit voltage (V_{oc}), short-circuit current (J_{sc}) and fill-factor (FF) can be observed in the fabricated device with Spiro-8Th used as the HTL compared to the fabricated device with Spiro-OMeTAD used as the HTL. The inventors monitored the long-term thermal and photo stability of devices with either Spiro-8Th or Spiro-OMeTAD as HTL.

FIG. 2 is a plot of Normalized PCE (%) versus elapsed time (hour) showing the photo-stability (at about 90±10 mW cm⁻²) for both the Spiro-8Th and the Spiro-OMeTAD, according to an embodiment of the present invention. The upper plot correspond to the Spiro-8Th and the lower plot corresponds to the Spiro-OMeTAD. The PCE of the Spiro-8Th is more photo stable over time and the PCE of the Spiro-8Th is higher than the PCE of the Spiro-OMeTAD. Devices with doped Spiro-8Th as HTL maintained 80.3% of its PCE for over 2000 h, while the PCE of Spiro-OMeTAD underwent through complete degradation after about 350 h, as shown in FIG. 2.

FIG. 3 is a plot of normalized PCE (%) versus elapsed time (hour) showing the thermal stability (at about 85° C.) for both the Spiro-8Th and the Spiro-OMeTAD, according to an embodiment of the present invention. The upper plot correspond to the Spiro-8Th and the lower plot corresponds to the Spiro-OMeTAD. The PCE of the Spiro-8Th is more thermally stable over time and is higher than the PCE the Spiro-OMeTAD. Devices with doped Spiro-8Th (dopants: TPFB: 4-Isopropyl-4′-methyldiphenyliodonium Tetrakis(pentafluorophenyl) borate) showed remarkable thermal stability at 85° C. under N₂ gas in glove box retaining 82.7% of its initial PCE for over 2000 h, while reference device with Spiro-OMeTAD completely degraded after 400 h, as shown in FIG. 3.

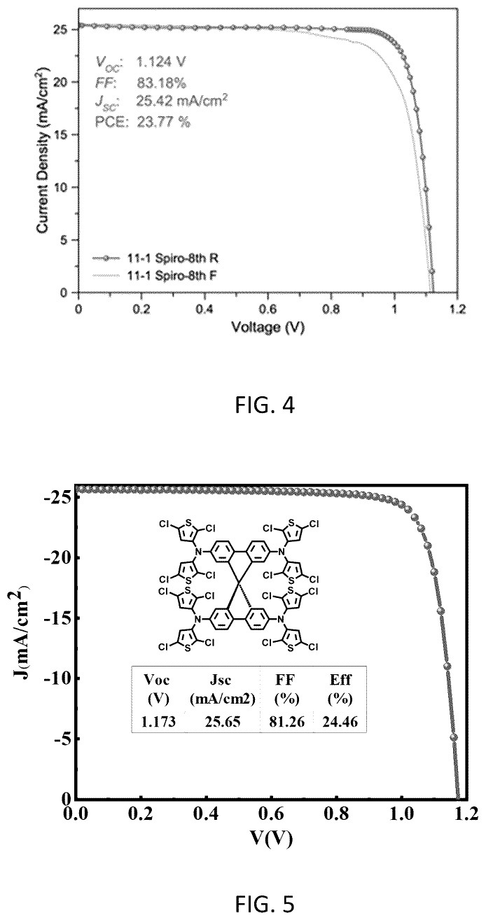

FIG. 4 is a plot of the current density (mA/cm2) versus the voltage (V) or (J-V) for both forward 11-1 Spiro-8Th F and reverse 11-1 Spiro-8Th R scan directions, according to an embodiment of the present invention. In this plot, the open circuit voltage (Voc) is set equal to 1.124 V, the fill factor (FF) is about 83.18%, the short-circuit current density (Jsc) is about 25.42 mA/cm², and the power conversion efficiency (PCE) is about 23.77%. FIG. 4 shows that the current density remains relatively constant over a broad range of voltage values, up to 1V. The plot shows a negligible hysteresis between forward and reverse scans, evidenced by the overlapping J-V curves, which suggests that the cell exhibits stable performance irrespective of the scan direction. This stable behavior is further supported by the cell's consistent key performance parameters: an open-circuit voltage (Voc) of 1.124 V, a fill factor (FF) of 83.18%, a short-circuit current density (J_{sc}) of 25.42 mA/cm², and a power conversion efficiency (PCE) of 23.77%. The close correspondence between the forward and reverse scans indicates minimal impact of ionic migration or charge trapping on the device's operation, highlighting the quality and effectiveness of the Spiro-8Th hole transport layer in perovskite solar cell applications.

In an embodiment, the Spiro-8Th can be synthesized using the following method. (a) A mixture of 2,2′,7,7′-tetraamino-9,9′-spirobifluorene (396.20 mg, 1.052 mmol), NaOt-Bu (2.22 g, 23.144 mmol), and Pd[P(t-Bu)₃]₂ (53.76 mg, 0.105 mmol) is added in toluene (100 mL). (b) 3-bromothiophene (3.43 g, 21.040 mmol) is subsequently added and the resulting mixture stirred at 100° C. for 48 h under argon atmosphere. (c) Upon cooling to room temperature, H₂O (100 mL) is added to the mixture, followed by extraction with dichloromethane (3×150 mL). (d) The organic phase is dried over MgSO₄ and concentrated under reduced pressure. (e) A purification of the product is achieved through column chromatography (silica gel, 3:2, hexane/dichloromethane), yielding a faint yellow solid as the product (1 g, 92%), which is further dissolved in a small amount of dichloromethane and recrystallized in methanol to obtain the Spiro-8Th material.

Characterization of Spiro-8Th using nuclear magnetic resonance (NMR) spectroscopy and high-resolution mass spectrometry (HRMS) is performed to confirm the molecular formula and measure the purity and quality of the obtained Spiro-8Th material. 1H NMR spectroscopy is performed on the Spiro-8Th in chloroform-d (CDCl3) at 298.5 K using a 400 MHz NMR spectrometer. This technique provided us with the chemical shifts (δ) and coupling constants (J) for the hydrogens in the molecule, which are indicative of the molecular structure and the environment of the hydrogen atoms within the Spiro-8Th compound. Additionally, 13C NMR spectroscopy is conducted in tetrachloroethane-d2 (C2D2C14) at 373.2 K with a 125 MHz spectrometer to identify the chemical shifts of the carbon atoms in the Spiro-8Th structure. The high-resolution mass spectrometry (HRMS) is performed using Matrix-Assisted Laser Desorption/Ionization (MALDI) with nitrogen as the matrix. The theoretical mass calculated for C57H36N4S8 is 1032.07001, and the measured mass is 1032.06922, which confirms the molecular formula and the high purity of the synthesized Spiro-8Th. These analytical techniques can be used for confirming the structure and purity of new chemical compounds such as Spiro-8Th, ensuring that the correct molecule is synthesized for application in devices such as perovskite solar cells.

¹H NMR (400 MHZ, CDCl3, 298.5 K) δ=7.55-7.53 (d, J=8.24 Hz, 4H), 7.18-7.16 (q, J₁=3.16 Hz, J₂=2 Hz, J₃=3.2 Hz, J₄=5.2 Hz, 8H), 7.03-7.01 (dd, J₁=8.32 Hz, J₂=2.1 Hz, 4H), 6.78-6.77 (dd, J₁=5.32 Hz, J₂=1.46 Hz, 8H), 6.65 (sd, J=1.96 Hz, 4H), 6.49-6.48 (sdd, J₁=3.24 Hz, J₂=1.44 Hz, 8H). ¹³C NMR (125 MHZ, C₂D₂Cl₄, 373.2 K): δ=150.11, 147.25, 147.02, 137.12, 124.77, 123.70, 120.41, 119.25, 110.16; HRMS (MALDI (N), 100%): calcd (%) for C₅₇H₃₆N₄S₈: 1032.07001; found, 1032.06922.

FIG. 5 is a plot of current density J (in mA/cm2) versus voltage (V) of a perovskite solar cell using Cl-substituted Spiro-8Th as the HTL, according to an embodiment of the present invention. As shown in FIG. 5, the current density versus voltage (J-V) characteristics of the perovskite solar cell with passivation layer employing Cl-substituted Spiro-8Th as the hole transport layer (HTL) showcases key performance metrics: 1) open-circuit voltage (Voc) at about 1.173 V, 2) short-circuit current density (Jsc) at about 25.65 mA/cm², 3) fill factor (FF) at about 81.26%, and 4) power conversion efficiency (PCE) at about 24.46%.

Building on inventors' foundation on the stability of carrier transport layers in perovskite solar cells (PSCs), studies are extended to include a Cl-substituted Spiro-8Th molecule as a hole transport layer (HTL) to provide non-crystalline molecules that dissolve readily in solutions and form dense, uniform films, which are beneficial for the efficiency and stability of PSCs.

The J-V characteristics of a perovskite solar cell utilizing Cl-substituted Spiro-8Th as the HTL exhibit promising key performance metrics, such as open-circuit voltage (Voc), short-circuit current density (Jsc), fill factor (FF), and power conversion efficiency (Eff). For example, the Cl substitution in Spiro-8Th is specifically configured and adapted to enhance the molecular stability against environmental stressors such as heat, humidity, and light exposure, which traditionally limit the practical application of perovskite solar cells.

By incorporating the Cl-substituted Spiro-8Th, this demonstrated that the present molecular design concept can be successfully applied to derivatives, affirming the validity of the present approach in synthesizing materials that not only improve the efficiency but also extend the operational lifespan of PSCs. This innovation paves the way for more stable and efficient perovskite solar cells, bringing us closer to the practical and widespread use of PSC technology.

The results achieved herein underscore the potential of Cl-substituted Spiro-8Th as an effective hole transport layer material that aligns with the high-performance and stability goals desired for advancing the field of perovskite solar cells.

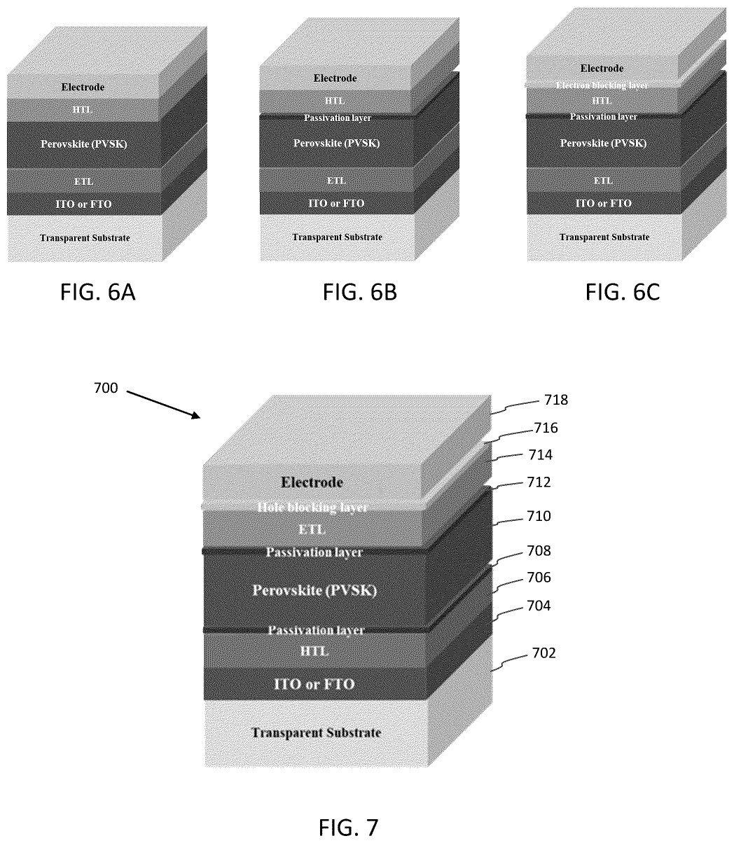

FIG. 6A is a schematic diagram of a basic structure of a perovskite solar cell highlighting the layered components from the transparent substrate to the electrode, according to an embodiment of the present invention.

FIG. 6B is a schematic diagram of a normal structure of a perovskite solar cell with an additional passivation layer to enhance performance and stability, according to another embodiment of the present disclosure.

FIG. 6C is a schematic diagram of a normal structure of a perovskite solar cell featuring both electron blocking and passivation layers for improved efficiency and longevity, according to yet another embodiment of the present invention.

FIG. 7 is a schematic diagram of an inverted structure of a perovskite (PVSK) device with hole-blocking layer and passivation layer, according to an embodiment of the present invention. In an embodiment, the inverted structure perovskite device uses passivation layer Al₂O₃ as the passivation layer to achieve 20.5% efficiency.

The PVSK device 700 includes a substrate 702 such as an indium tin oxide (ITO) substrate. An indium tin oxide (ITO) layer or fluoride tin oxide (FTO) layer 704 is deposited on the substrate 702. The substrate 702 is used as a first electrode. A hole transport layer (HTL) 706 is deposited on the ITO or FTO layer 704. A PVSK active layer 710 is sandwiched between passivation layer 708 and passivation layer 712 and deposited on the HTL706. An electron transport layer (ETL) 714 is deposited on passivation layer 712. A hole blocking layer 716 is provided on top of the ETL layer 714. A second electrode layer 718 (e.g., silver or gold layer) is deposited on the hole blocking layer 716. In an embodiment, the passivation layers 708 and 712 include Al₂O₃ so that the PVSK solar cell 700 achieves 20.5% efficiency. In an embodiment, the HTL 706 includes the Spiro-8Th molecule or the Spiro-8Th polymer.

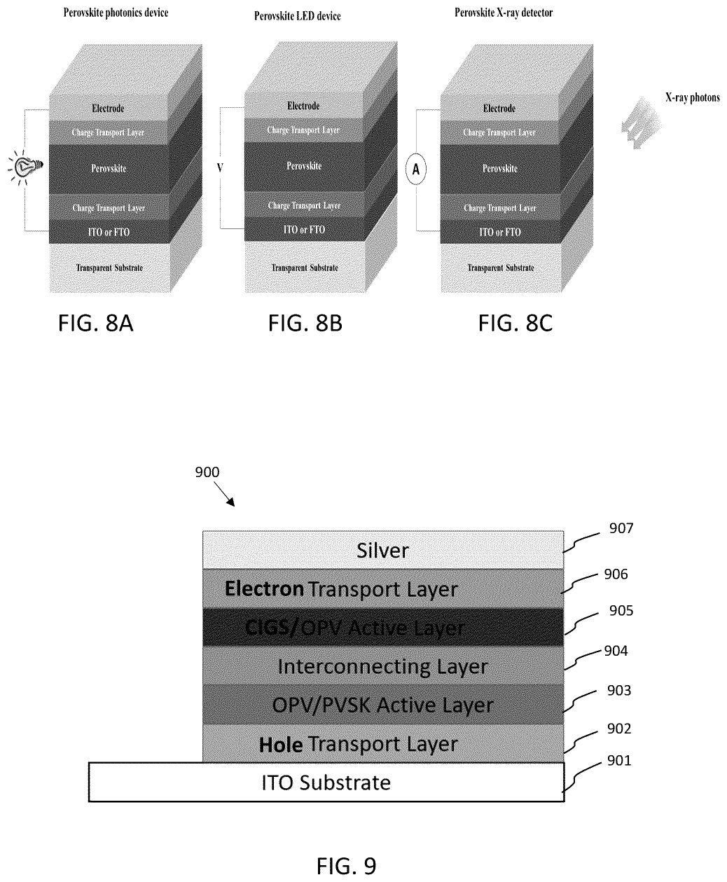

In an embodiment, the PVSK device 700 can be a solar cell, a light emitting device or an organic photodetector (OPD) to detect radiation (IR, VIS, UV, X-Rays). FIG. 8A is a schematic diagram of perovskite photonics device using a charge transport layer based on Spiro-8Th, according to an embodiment of the present invention. FIG. 8B is a schematic diagram of perovskite light emitting device (LED) using a charge transport layer based on Spiro-8Th, according to an embodiment of the present invention. FIG. 8C is a schematic diagram of perovskite X-ray detector using a charge transport layer based on Spiro-8Th, according to an embodiment of the present invention. FIGS. 8A, 8B and 8C illustrate the versatile applications of a perovskite-based charge transport layer (HTL) in photonic devices, light emitting devices (LEDs), and X-ray detecting devices, highlighting the adaptability of such perovskite-based charge transport layer (HTL) for use in diverse optoelectronic contexts. The device structure includes normal structure and inverted structure.

FIG. 9 is schematic diagram of a structure of a tandem device 900 using Spiro-8Th in the hole transport layer, according to an embodiment of the present invention. The tandem device 900 (e.g., a solar cell) includes a substrate 601 such as an indium tin oxide (ITO) substrate. The substrate 601 is used as a first electrode. A hole transport layer (HTL) 602 is deposited on the substrate 601. The HTL 602 may include the Spiro-8Th material. A first organic photovoltaic (OPV) active layer 903 is deposited on the hole transport layer (HTL) 602. The first OPV active layer 903 can be a PVSK layer. An interconnecting layer 604 is deposited on the first OPV active layer 903. A second OPV active layer 905 is deposited on the interconnecting layer 604. The second OPV active layer 905 can be a copper indium gallium selenide (CIGS) layer, a silicon (Si) layer, or a PVSK layer. An electron transport layer (ETL) 906 is deposited on the second OPV active layer 905. The ETL may also include the Spiro-8Th material. A metal layer 907 is deposited on the ETL 906. The metal layer 907 (e.g., silver layer) is used as a second electrode. In an embodiment, one or more passivation layers (not shown in FIG. 9 but shown in FIG. 7) can be provided in contact with the first OPV (e.g., PVSK) active layer 903 and/or the second OPV (e.g., CIGS) active layer 905. In an embodiment, a hole blocking layer (not shown in FIG. 9 but shown in FIG. 7) can also be provided between the ETL 906 and the second electrode 907.

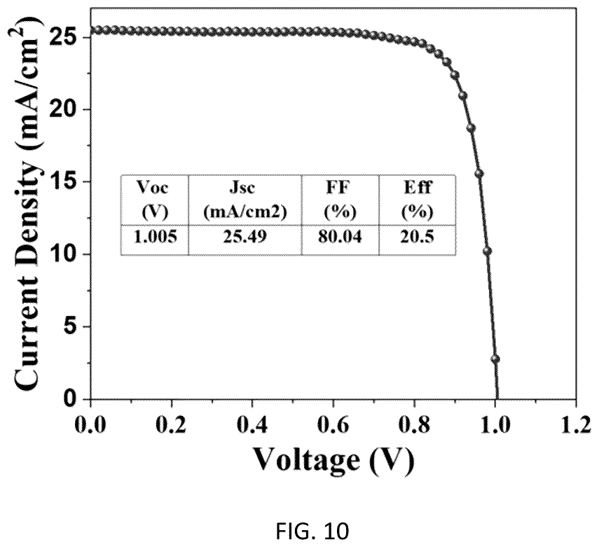

FIG. 10 is a plot of current density J (in mA/cm2) versus voltage (V) of a perovskite solar cell using Cl-substituted Spiro-8Th as the HTL, according to an embodiment of the present invention. The current density versus voltage (J-V) characteristics of a perovskite solar cell Spiro-8Th as the hole transport layer, shown in FIG. 10, showcases key performance metrics: open-circuit voltage (Voc) at about 1.005 V, short-circuit current density (Jsc) at about 25.49 mA/cm², fill factor (FF) at about 80.04%, and a power conversion efficiency (PCE) at about 20.5%.

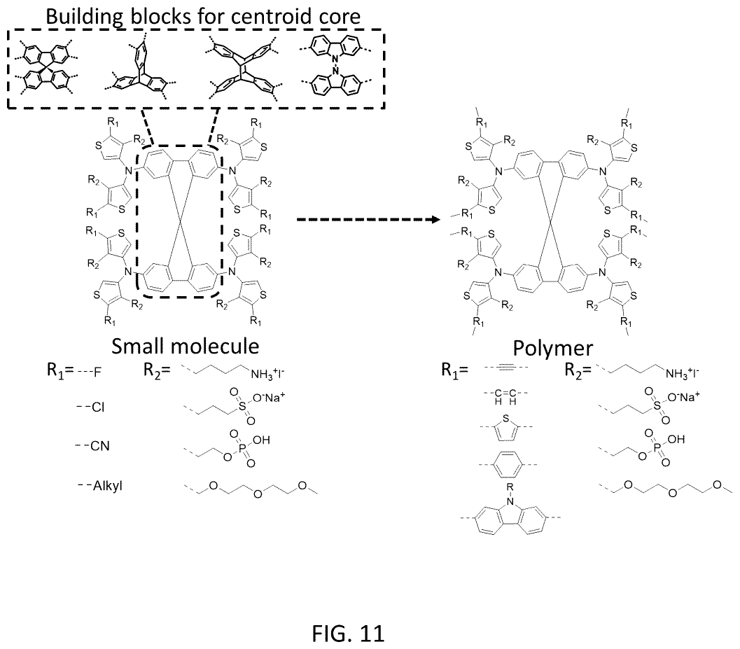

FIG. 11 shows examples of a small molecule spiro-8Th and polymer molecule Spiro-8Th in HTL which substituents can be tailored to meet desired applications, according to embodiments of the present invention.

In the above paragraphs, the Spiro-8Th is described being used in the hole transport layer (HTL). However, the Spiro-8Th can also be used in an electron transport layer (ETL) or both the HTL and the ETL.

An aspect of an embodiment of the present invention is to provide a spiro-thiophene compound including a centroid core having a plurality of benzene rings; and a plurality of thiophene molecules covalently bonded to a carbon atom of the benzene rings of the centroid core to form a centroid-thiophene molecule.

In an embodiment, the plurality of centroid-thiophene molecules of the spiro-thiophene compound are cross-linked to form a spiro-thiophene polymer.

In an embodiment, the spiro-thiophene polymer provides enhanced impedance of ion-migration.

In an embodiment, the centroid core of the spiro-thiophene compound includes two spirobifluorene (SF) units forming a rigid biplanar conjugated cruciform-shaped spirobifluorene.

In an embodiment, the two spirobifluorene (SF) units forming the rigid biplanar conjugated cruciform-shaped spirobifluorene are selected from the group consisting of:

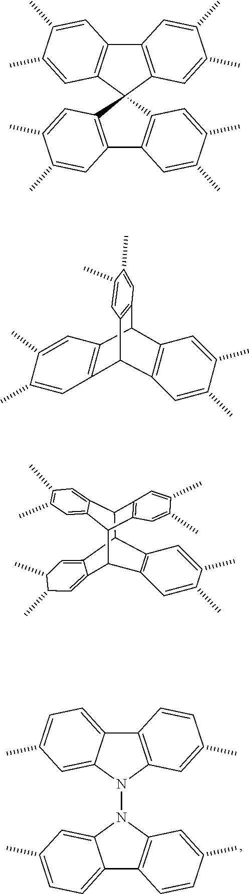

In an embodiment, the spiro-thiophene compound further includes an R1-group bonded to one carbon of at least one benzene ring to increase solvent resistance and to strengthen intermolecular interactions and increase hole mobility, wherein the R1-group is selected from the group consisting of F, Cl, CN, and alkyl.

In an embodiment, the spiro-thiophene compound further includes an R2-group bonded to another one carbon of the at least one benzene ring to control a hydrophobicity and a hydrophilicity of the spiro-thiophene compound, wherein the R2 group is selected from the group consisting of:

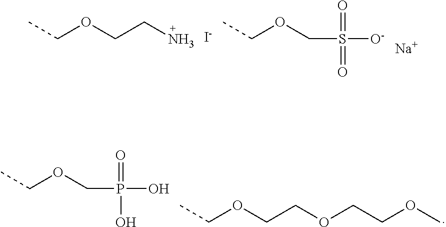

In an embodiment, the plurality of thiophene molecules are covalently bonded to a carbon atom of the benzene rings of the centroid core to form the centroid-thiophene molecule as follows:

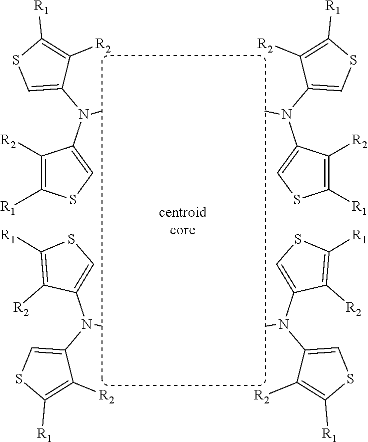

wherein R1 and R2 are any one of the following radicals:

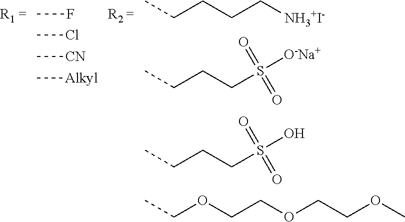

In an embodiment, the plurality of centroid-thiophene molecules are cross-linked to form the spiro-thiophene as follows:

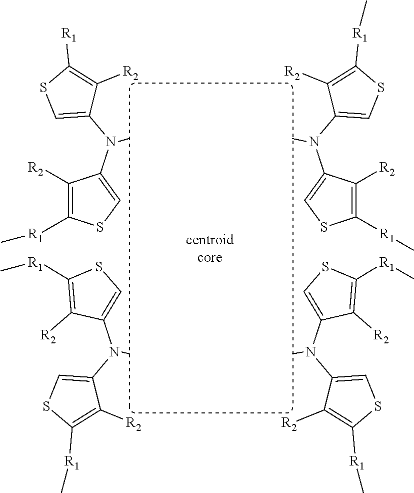

wherein R1 and R2 are selected from the group consisting of:

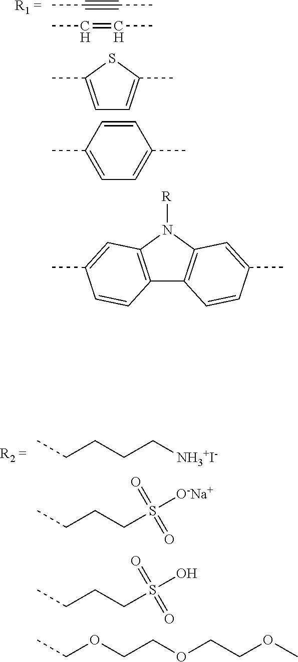

In an embodiment, the plurality of centroid-thiophene molecules are cross-linked when the R1-group is replaced by a cross-linker selected from the group consisting of:

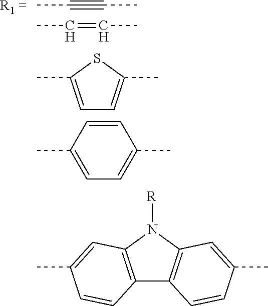

In an embodiment, the plurality of centroid-thiophene molecules are cross-linked using thermal annealing or UV exposure induced radical polymerization reaction.

In an embodiment, the spiro-thiophene compound has a hole mobility greater than a mobility in spiro-OMeTAD for use as high-performance hole-transporting layer including N-I-P and P-I-N device architectures having a sequence of hole-transport layer (p), intrinsic absorber layer (i), and electron-transport layer (n).

In an embodiment, the spiro-thiophene compound has a more amorphous morphology than a morphology of Spiro-OMeTAD so as to improve a stability of the spiro-thiophene compound when used in a device.

In an embodiment, the spiro-thiophene and its derivatives are used as a buffer layer to suppress ion migration to improve stability of a device using the spiro-thiophene compound.

In an embodiment, the spiro-thiophene and its derivatives are as used as a hole transport layer (HTL) or used as an interfacial layer for tandem device fabrication.

In an embodiment, the spiro-thiophene and its derivatives are used as an electron transport layer (ETL). A precursor of spiro-thiophene can be further synthesized into ETL imide materials using aromatic anhydride reactant. The chemical formula of the spiro-thiophene material when used ETL imide materials is as follows:

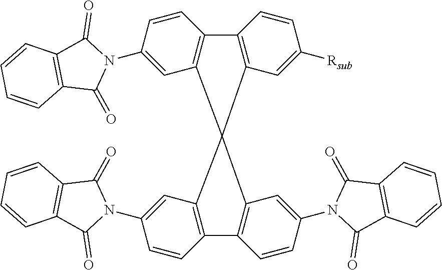

wherein the substituent radical R_{sub} is selected from the group consisting of:

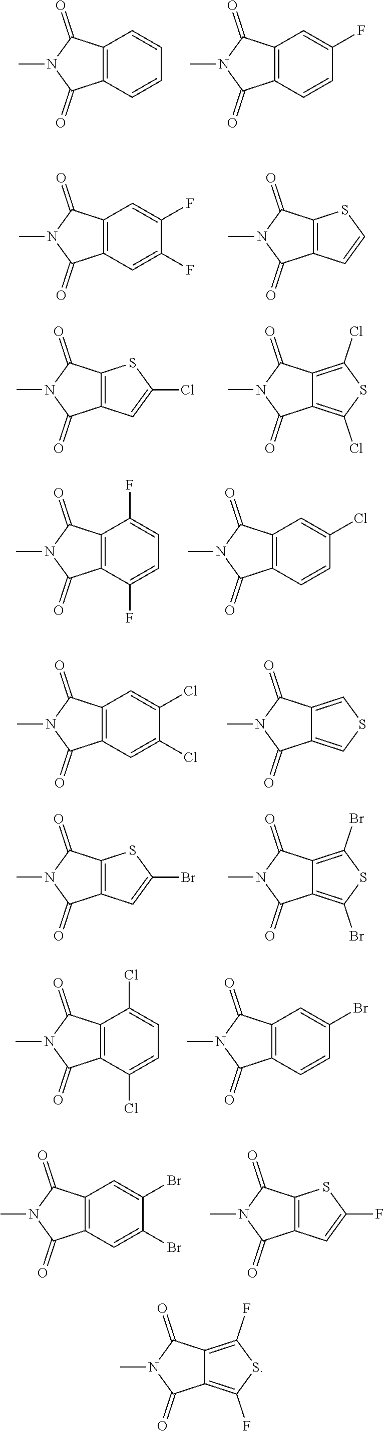

In an embodiment, the tandem device includes perovskite-perovskite tandem device, perovskite-silicon tandem device, or perovskite-Copper Indium Gallium Selenide (perovskite-CIGS) tandem device.

An aspect of the present invention is to provide a perovskite (PVSK) structure having the spiro-thiophene compound described in the above paragraphs.

In an embodiment, the PVSK structure is formed via either surface coating on top of a PVSK material, or by blending the spiro-thiophene compound within a perovskite solution.

In an embodiment, the spiro-thiophene compound is doped. In an embodiment, the dopant includes but not limited to the group consisting of an organic salt (-Isopropyl-4′-methyldiphenyliodoniumTetrakis(pentafluorophenyl) borate (TPFB), derivatives of TPFB, organic compound (Tetracyanoquinodimethane (TCNQ), derivatives of TCNQ, 4-tert-butyl-1-methylpyridinium bis(trifluoromethylsulfonyl)imide (TBMP+TFSI−).

In an embodiment, the dopant includes inorganic dopants, such as lithium salts (Lithium Tetrafluoroborate or Lithium Bis(trifluoromethanesulfonyl)imide) and Cobalt salts (Cobalt (II) Phthalocyanine or Cobalt (III) Tetraphenylporphyrin).

In an embodiment, the inorganic dopant is selected from the group consisting of rare-earth elements.

Another aspect of the present invention is to provide a method of producing a spiro-thiophene compound. The method includes:
(a) providing an initial mixture of 2,2′,7,7′-tetraamino-9,9′-spirobifluorene, NaOt-Bu, and Pd[P(t-Bu)3]2;
(b) adding 3-bromothiophene to the initial mixture to provide a subsequent mixture;
(c) stirring the subsequent mixture under inert atmosphere;
(d) cooling the subsequent mixture to room temperature;
(e) adding water to the subsequent mixture;
(f) extracting with dichloromethane to provide an organic phase;
(g) drying the organic phase over MgSO₄ and concentrating under reduced pressure;
(h) purifying the organic phase through column chromatography to provide a solid as a product comprising spiro-thiophene molecules,
(i) dissolving the product in dichloromethane and recrystallizing in methanol.

In an embodiment, the method further includes, prior to adding 3-bromothiophene to the initial mixture, adding toluene to the initial mixture.

A further aspect of the present invention is to provide a spiro-thiophene compound produced using the above method.

Another aspect of the present invention is to provide a method of producing a polymer of spiro-thiophene. The method includes cross-linking a plurality of spiro-thiophene molecules produced with the above method.

In an embodiment, cross-linking the plurality of spiro-thiophene molecules includes cross-linking the plurality of spiro-thiophene molecules using a cross-linker, using thermal annealing, or using UV exposure induced radical polymerization reaction.

Another aspect of the present invention is to provide a spiro-thiophene polymer produced using the above method of producing the polymer of spiro-thiophene.

Another aspect of the present invention is to provide a perovskite device comprising a hole transport layer (HTL) having a spiro-thiophene compound including a centroid core having a plurality of benzene rings, and a plurality of thiophene molecules covalently bonded to a carbon atom of the plurality of benzene rings of the centroid core to form a centroid-thiophene molecule.

Another aspect of the present invention is to provide a device including a first electrode and a second electrode; a first organic photovoltaic layer provided between the first electrode and the second electrode; and a hole transport layer provided between the first organic photovoltaic layer and the first electrode or between the first organic photovoltaic layer and the second electrode. The hole transport layer has a spiro-thiophene compound comprising a centroid core having a plurality of benzene rings, and a plurality of thiophene molecules covalently bonded to a carbon atom of the plurality of benzene rings of the centroid core to form a centroid-thiophene molecule.

In an embodiment, the first organic photovoltaic layer is a perovskite layer.

In an embodiment, the device further includes a second organic photovoltaic layer provided between the first electrode and the second electrode so as to form a tandem device.

In an embodiment, the second organic photovoltaic layer is a silicon layer, a perovskite layer, or a CIGS layer.

In an embodiment, the device further includes one or more passivation layers provided in contact with the first organic photovoltaic layer.

In an embodiment, the device is at least one of a photonics device, a light emitting device, or an X-ray detecting device.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A spiro-thiophene compound comprising:
a centroid core having a plurality of benzene rings; and
a plurality of thiophene molecules covalently bonded to a carbon atom of the plurality of benzene rings of the centroid core to form a centroid-thiophene molecule.

2. The spiro-thiophene compound according to claim 1, wherein a plurality of centroid-thiophene molecules are cross-linked to form a spiro-thiophene polymer.

3. The spiro-thiophene compound according to claim 2, wherein the spiro-thiophene polymer provides enhanced impedance of ion-migration.

4. The spiro-thiophene compound according to claim 1, wherein the centroid core comprises two spirobifluorene (SF) units forming a rigid biplanar conjugated cruciform-shaped spirobifluorene.

5. The spiro-thiophene compound according to claim 4, wherein the two spirobifluorene (SF) units forming the rigid biplanar conjugated cruciform-shaped spirobifluorene are selected from the group consisting of:

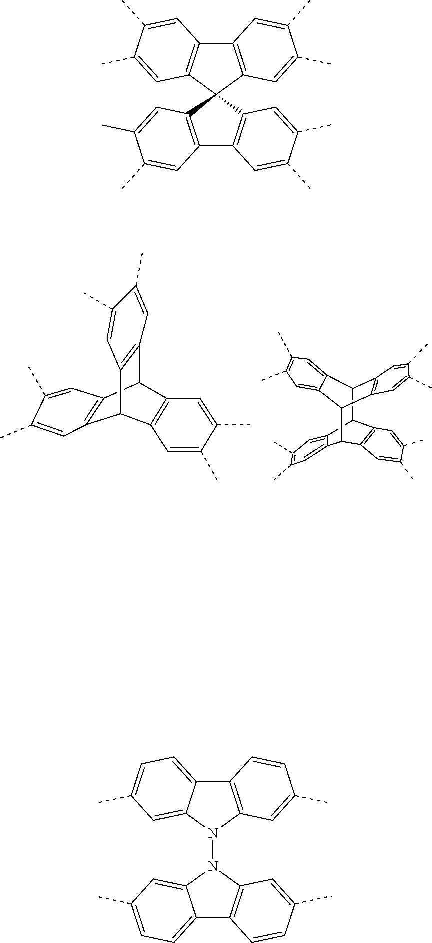

6. The spiro-thiophene compound according to claim 5, further comprising an R1-group bonded to one carbon of at least one benzene ring to increase solvent resistance and to strengthen intermolecular interactions and increase hole mobility, wherein R1 is selected from the group consisting of F, Cl, CN, and alkyl.

7. The spiro-thiophene compound according to claim 6, further comprising an R2-group bonded to another one carbon of the at least one benzene ring to control a hydrophobicity and a hydrophilicity of the spiro-thiophene compound, wherein the R2-group is selected from the group consisting of:

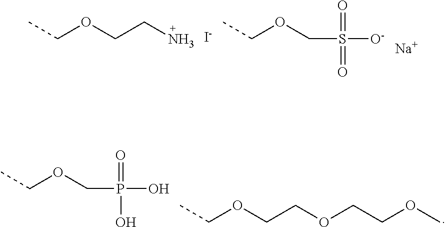

8. The spiro-thiophene compound according to claim 5, wherein the plurality of thiophene molecules are covalently bonded to a carbon atom of the benzene rings of the centroid core to form the centroid-thiophene molecule as follows: 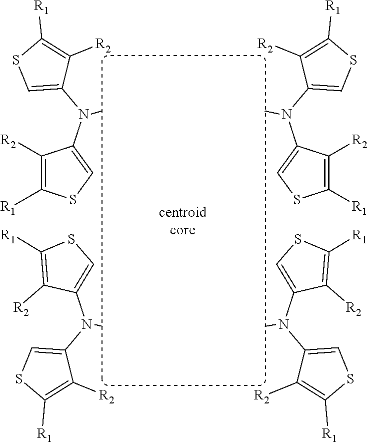
wherein R₁ and R₂ are any one of the following radicals:

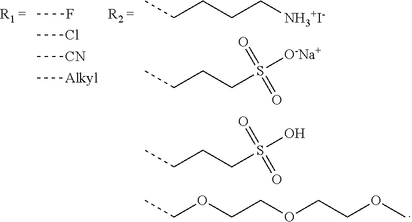

9. The spiro-thiophene compound according to claim 8, wherein the plurality of thiophene molecules are cross-linked to form the spiro-thiophene compound as follows: 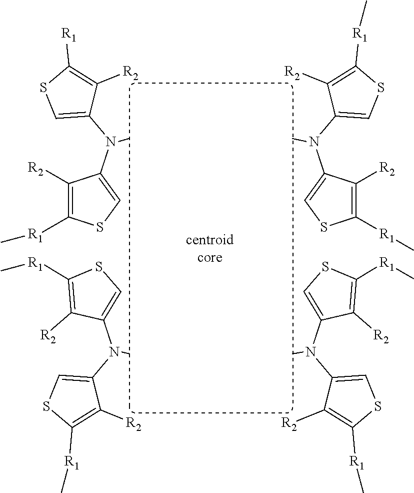
wherein R₁ and R₂ are selected from the group consisting of:

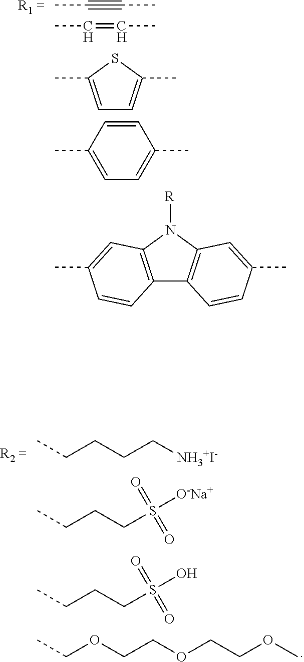

10. The spiro-thiophene compound according to claim 9, wherein the plurality of thiophene molecules are cross-linked when the R₁-group is replaced by a cross-linker selected from the group consisting of:

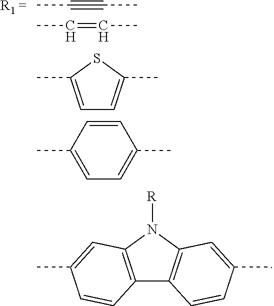

11. The spiro-thiophene compound according to claim 1, the spiro-thiophene compound having the following chemical formula: 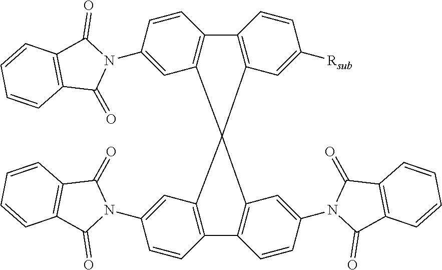
wherein the substituent radical R_{sub} is selected from the group consisting of:

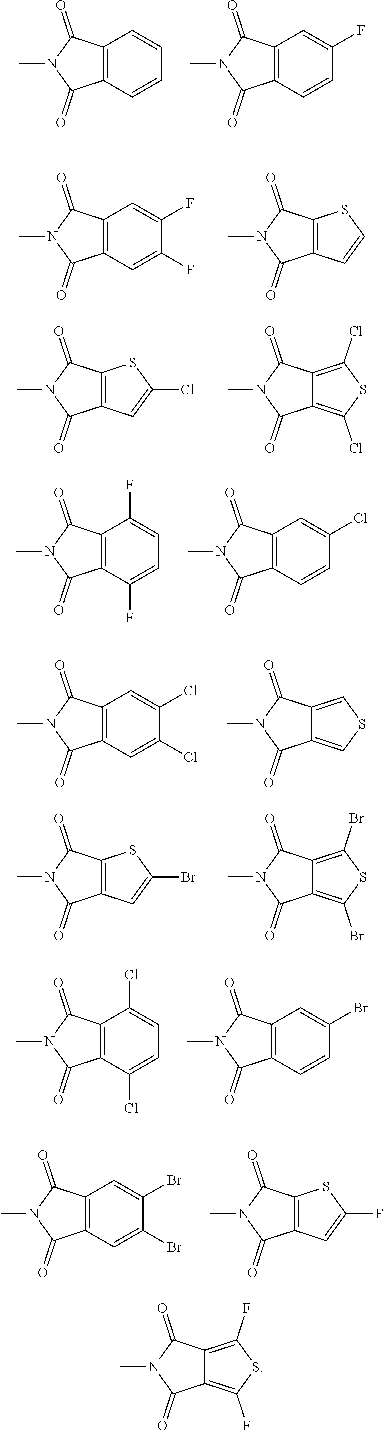

12. The spiro-thiophene compound according to claim 1, wherein the spiro-thiophene compound has a hole mobility greater than a mobility in spiro-OMeTAD for use as high-performance hole-transporting layer including N-I-P, P-I-N device architectures having a sequence of hole-transport layer (p), intrinsic absorber layer (i), and electron-transport layer (n).

13. The spiro-thiophene compound according to claim 1, wherein the spiro-thiophene compound has a more amorphous morphology than a morphology of spiro-OMeTAD so as to improve a stability of the spiro-thiophene compound when used in a device.

14. A perovskite (PVSK) structure comprising the spiro-thiophene compound according to claim 1.

15. The perovskite (PVSK) structure according to claim 14, wherein the structure is formed via either surface coating on top of a PVSK material, or by blending the spiro-thiophene compound within a perovskite solution.

16. The perovskite (PVSK) structure according to claim 14, wherein the spiro-thiophene compound is doped.

17. The perovskite (PVSK) structure according to claim 16, wherein a dopant is selected from the group consisting of an organic salt (-Isopropyl-4′-methyldiphenyliodoniumTetrakis(pentafluorophenyl) borate (TPFB), derivatives of TPFB, organic compound (Tetracyanoquinodimethane (TCNQ), derivatives of TCNQ, 4-tert-butyl-1-methylpyridinium bis(trifluoromethylsulfonyl)imide (TBMP⁺TFSI⁻) and inorganic dopants, such as lithium salts (Lithium Tetrafluoroborate or Lithium Bis(trifluoromethanesulfonyl)imide) and Cobalt salts (Cobalt (II) Phthalocyanine or Cobalt (III) Tetraphenylporphyrin).

18. The perovskite (PVSK) structure according to claim 16, wherein a dopant is selected from the group consisting of Lithium Tetrafluoroborate, Lithium Bis(trifluoromethanesulfonyl)imide, Cobalt (II) Phthalocyanine, Cobalt (III) Tetraphenylporphyrin, and rare-earth elements.

19. A method of producing a spiro-thiophene compound, the method comprising:
providing an initial mixture of 2,2′,7,7′-tetraamino-9,9′-spirobifluorene, NaOt-Bu, and Pd[P(t-Bu)₃]₂;
adding 3-bromothiophene to the initial mixture to provide a subsequent mixture;
stirring the subsequent mixture under inert atmosphere;
cooling the subsequent mixture to room temperature;
adding water to the subsequent mixture;
extracting with dichloromethane to provide an organic phase;
drying the organic phase over MgSO₄ and concentrating under reduced pressure;
purifying the organic phase through column chromatography to provide a solid as a product comprising spiro-thiophene molecules; and
dissolving the product in dichloromethane and recrystallizing in methanol to produce the spiro-thiophene compound.

20. The method according to claim 19, further comprising prior to adding 3-bromothiophene to the initial mixture adding toluene to the initial mixture.

21. The method of according to claim 19, further comprising cross-linking a plurality of spiro-thiophene molecules.

22. The method according to claim 21, wherein cross-linking the plurality of spiro-thiophene molecules comprises cross-linking the plurality of spiro-thiophene molecules using a cross-linker, using thermal annealing, or using ultraviolet exposure induced radical polymerization reaction.

23. A device comprising:
a first electrode and a second electrode;
a first organic photovoltaic layer provided between the first electrode and the second electrode; and
a hole transport layer provided between the first organic photovoltaic layer and the first electrode or between the first organic photovoltaic layer and the second electrode,
wherein the hole transport layer has a spiro-thiophene compound comprising a centroid core having a plurality of benzene rings, and a plurality of thiophene molecules covalently bonded to a carbon atom of the plurality of benzene rings of the centroid core to form a centroid-thiophene molecule.

24. The device according to claim 23, wherein the first organic photovoltaic layer is a perovskite layer.
